(19) United States
(12) Patent Application Publication
(10) Pub. No.: US 2026/0096916 A1
(43) Pub. Date: Apr. 9, 2026

(54) OSTOMY APPLIANCE

(71) Applicants: Thomas R. Galbierz, Brentwood, TN (US); Michael A. Galbierz, St. Louis, MO (US)

(72) Inventors: Thomas R. Galbierz, Brentwood, TN (US); Michael A. Galbierz, St. Louis, MO (US)

(21) Appl. No.: 19/349,631

(22) Filed: Oct. 3, 2025

Related U.S. Application Data
(60) Provisional application No. 63/703,690, filed on Oct. 4, 2024.

(51) Int. Cl. A61F 5/445; A61F 5/448; A61L 24/00; A61L 28/00

(52) U.S. Cl. CPC A61F 5/445; A61L 24/00; A61L 28/0003; A61F 2005/4486

(57) ABSTRACT

An ostomy appliance comprising a barrier and a pouch which is adhered to the barrier. The barrier is made of a material comprising a top layer, a reinforcing layer, and an adherent layer. The top layer comprises a base material and upper and lower piles extending from opposite surfaces of said base material. The reinforcing layer being secured to said top layer by a first binder which will bond to said reinforcing layer. The adherent layer comprising a silicone gel which is bonded to said reinforcing layer by a second binder.

### CROSS-REFERENCE TO RELATED APPLICATIONS

This application claims priority to U.S. App. No. 63/703,690 filed Oct. 4, 2024 which is entitled “Ostomy Appliance” and which is incorporated herein by reference.

### STATEMENT REGARDING FEDERALLY SPONSORED RESEARCH OR DEVELOPMENT

Not Applicable.

### BACKGROUND

This application relates generally to ostomy appliances or ostomy bags/pouches.

As is known, there are two types of ostomies-loop ostomies, when a loop of, for example, colon or intestine, is pulled through an opening in the abdomen and opened up to form a loop stoma, and an end ostomy, when, for example, the end of the colon or small intestine extends through an opening in the abdomen to form an end stoma. Ostomy appliances surround and enclose the stoma to collect output from the stoma. Ostomy appliances comprise an ostomy bag or pouch which is secured to the top side or surface of a base or flange, which, in turn, is secured to the abdomen around the ostomy by means of an adhesive applied to the backing of the base. The base has a hole sized to surround the wearer's stoma and which is aligned with an opening into the bag to thereby place the stoma in communication with the interior of the bag. Typically, the base will be formed from a material which can be cut, so that the hole can be formed to the appropriate size for the stoma to reduce the amount of peristomal skin exposed between the stoma and the perimeter of the hole. Waste, such as fecal matter, thus passes from the stoma into the bag. This stoma-receiving opening is near the top of the bag on a back surface of the bag. The bag usually also includes a second opening at its bottom which allows for the bag to be emptied without the need to remove the bag from the base.

FIGS. 1A and 1B are schematic vertical cross-sectional views of generic one-piece and two-piece ostomy appliances, respectively. Both the one-piece and two-piece ostomy appliances A1, A2 comprise a flange or base portion 1 which adheres to the wearer's skin and defines an opening 3 which is selectively sized to fit about the wearer's stoma. An ostomy bag 5 is then secured to the base or flange 1. The ostomy bag 5 includes an opening 7 in alignment with the opening 3 in the appliance base such that the stoma will extend into the interior of the bag, so that output from the stoma (colon, ileus, or urethra) will be deposited in the bag. In both the one-piece and two-piece appliance, an accumulation volume 8 of the ostomy appliance is defined fully by the ostomy bag 5. In the one-piece appliance A1, the bag 5 is permanently fixed to the base 1 and there is a narrow connection or neck area 9 at the junction of the bag 5 and base 1. In a two-piece appliance A2, the base 1 and bag 5 will each have a ring R1, R2 surrounding their respective openings. The two rings are sized and shaped to be matingly connected, to thus (removably) connect the bag to the flange. In this instance, the rings R1,R2, in combination, can define a short neck. The outer circumference of the necks/connectors of the one- and two-piece ostomy appliances is typically in the range of about 2-4 inches. The openings 3,7 through which the stoma extends can have a diameter of between 10 mm (˜0.4″) and 60 mm (˜2.4″) and is sized according to the size of the stoma.

The base 1 must be sized, as noted above, to be applied to a stoma. Sizing comprising enlarging the opening 3 as necessary so that the opening 3 will fit about the stoma. Depending on how the base 1 is made, the size of the opening 3 can be fixed or the base material can allow for sizing of the opening (for example, by cutting the material to increase the size of the opining 3). Ideally, the opening 3 will be sized such that the edge of the opening 3 will abut the stoma. This reduces the potential for stomal output from contacting (and irritating) skin adjacent the stoma and under the base 1. The base is formed from a soft material to reduce the potential for the base to irritate the stoma. However, the base can be thick, making it difficult to size.

Typically, the base is adhered to the skin by means of a hydrocolloid layer or adhesive. This hydrocolloid layer provides a relatively weak adherence to the skin, to reduce damage to the skin when the appliance is removed. However, the hydrocolloid layer is moisture retentive, and can thus absorb liquid from the stomal output. This can lead to irritation of the skin if the ostomy appliance is not changed regularly.

Additionally, the seal of the base around the stoma is not always sufficient, and ostomy output (which can be acidic and/or corrosive, especially for an ileostomy) can contact the skin around the stoma, causing the peristomal skin to become irritated or inflamed. Sealing problems can occur, for example, when the stoma is sunken relative to the abdominal surface or when the skin surrounding the stoma (the peristomal plane) is not flat, such as when there are folds or creases in the skin. Such physiology can create an area around the stoma that is sunken relative to the rest of the abdomen, making it difficult for the base of the appliance to seal properly against the wearer's skin and can leave gaps or cavities into which bodily fluids can leak. Various manufacturers have attempted to solve this issue by providing different configurations of bases and different methods for mounting the bag to the base.

It would be desirable to provide a product that can overcome these issues and which would provide an improved ostomy appliance.

### BRIEF DESCRIPTION OF THE DRAWINGS

FIGS. 1A-B comprise schematic vertical cross-sectional views of (A) a one-piece and (B) a two-piece ostomy appliance;

FIG. 2 is an enlarged schematic cross-sectional view of the material from which the barrier of the ostomy appliance is made;

FIG. 3A is a schematic cross-sectional view of a seal between a pouch and barrier of a first one-piece ostomy appliance;

FIG. 3B is a schematic cross-sectional view of the first one-piece ostomy appliance;

FIG. 3C is a schematic plan view of the first one-piece ostomy appliance showing the ostomy bar or pouch and the barrier;

FIG. 4A is a schematic cross-sectional view of a seal between the pouch and barrier of a second one-piece ostomy appliance;

FIG. 4B is a schematic plan view of the one-piece ostomy appliance of FIG. 4A showing the ostomy bar or pouch and the barrier;

FIG. 5 is a schematic cross-sectional views of a seal between the pouch and barrier of a first two-piece ostomy appliance;

FIG. 6 is a schematic, exploded cross-sectional view of the seal between the pouch and barrier of the two-piece ostomy appliance of FIG. 5;

FIG. 7 is an enlarged view taken at circle 7 of FIG. 6;

FIG. 8 is a schematic cross-sectional view of a seal between the pouch and barrier of a second two-piece ostomy appliance;

FIG. 9 is a cross-sectional view of a third two-piece ostomy appliance;

FIGS. 10A-D are cross-sectional views of a one-piece ostomy appliance generally similar to that of FIG. 1, but showing 4 different ways to bond the ostomy appliance pouch to the ostomy appliance barrier.

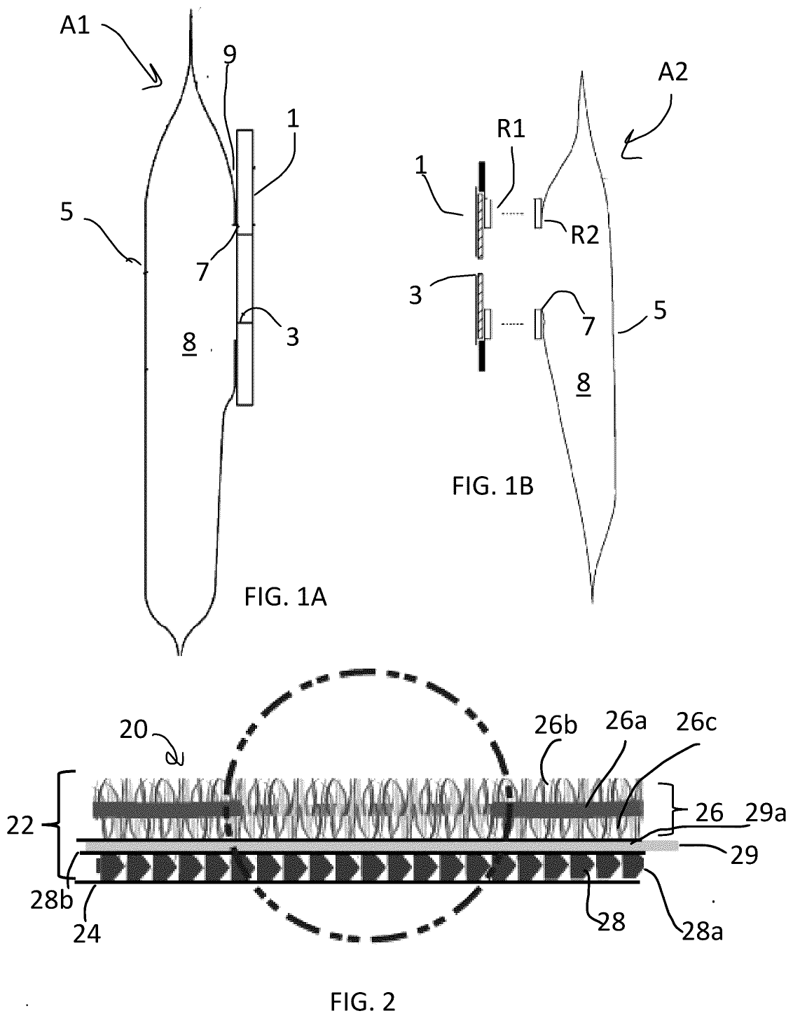

Corresponding reference numerals will be used throughout the several figures of the drawings.

### DETAILED DESCRIPTION

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what we presently believe is the best mode of carrying out the claimed invention. Additionally, it is to be understood that the claimed invention is not limited in its application to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

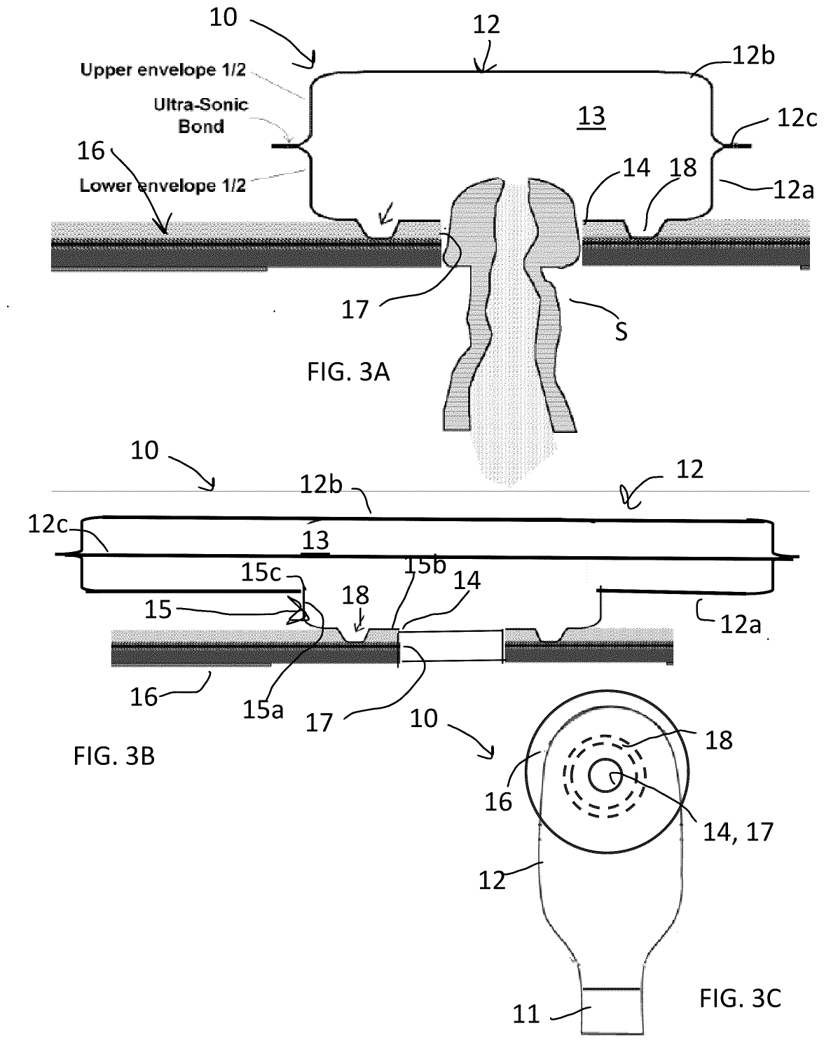

A one-piece ostomy appliance 10 is shown in FIGS. 3A-C. The ostomy appliance 10 comprises an ostomy pouch 12 comprised of a lower envelope member 12a and an upper envelope member 12b which are joined together along a seam 12c at their respective peripheries to, in combination, define an accumulation volume 13. The lower and upper envelope members 12a,b can, for example, be ultrasonically bonded together along to form the seam 12c. They can also be joined via heat sealing, adhesive, or any other desired manner that will enable the upper and lower envelope members to define the accumulation volume 13. The pouch 12 can have a bottom 11 (FIG. 3C) that is open or openable to allow for emptying of the pouch 12.

A connection assembly 15 extends from lower envelope member 12a. The connection assembly 15 comprises an annular neck or wall 15a and a flange 15b at a bottom of the wall 15a. As best seen in FIG. 3B, the flange extends inwardly from the wall 15a to define an opening 14. As seen, the opening 14 is smaller in diameter (and preferably concentric with) the junction 15c between the connection assembly 15 and the pouch lower envelope member 12a. The opening 14 is sized to surround a stoma S.

The lower envelope 12a is adhered via the connection assembly 15 to a barrier 16 having an opening 17 through which the stoma S can extend. The barrier opening 17 and pouch opening 14 are aligned, as seen in FIGS. 3A-C. The material 20 from which the barrier is made is shown schematically in cross-section in FIG. 3. This material is substantially the same material as disclosed in PCT/US2024/0260004, which is incorporated herein by reference. The material 20 is an adhesive assembly comprising an elastic, stretchable and flexible barrier material 22 and a release liner 24. The barrier material 22 comprises a top layer 26, a reinforcing layer 29, and an adherent layer 28. The release liner 24 is removably secured to the adherent layer 28 of the barrier material 22 to protect the adherent layer 28 until the barrier material is put into use. When the release liner 24 is removed, the adherent layer 28 will removably adhere an ostomy appliance barrier 16 formed from the barrier material 22 to a surface, such as a patient's skin.

The top layer 26 preferably comprises a stretchable, elastic base material 26a which can be formed from a knit material, a woven material, or a tricot material. Preferably the top layer 26 is elastic and stretchable in all directions, rather than in just one direction. The material which forms the base material 26a can comprise man-made fibers, natural fibers, or a combination thereof. Preferably, the base material 26a is formed from a combination of fibers, at least a portion of which are elastic fibers, such as spandex (a polyether-polyurea copolymer), such as, for example, Lycra®. If formed from a combination of fibers, the elastic fibers can be combined, intertwined, or formed with, for example, natural fibers, such as cotton fibers, or man-made fibers, such as nylon or spandex, to form the thread or yarn from which the material for the top layer 26 is formed. The elastic fibers provide the stretchability and elasticity of the top layer 26. The base material can, for example, be about 5% to about 95% nylon and about 95% to about 5% spandex. In a preferred embodiment, the base material is about 90% nylon and about 10% spandex. Illustratively, the nylon can be SD Nylon and the spandex can be 40 Denier Spandex. Further, the material can comprise different types of nylon, such as “50/17 SD” nylon and “20/1 SD” nylon. The two types of nylon can be combined in ratios of about 4:1 “50/17 SD” Nylon: “20/1 SD” Nylon to about 1:4 “50/17 SD” Nylon: “20/1 SD” Nylon, and preferably about 3.2:1 “50/17 SD” Nylon: “20/1 SD” Nylon. In a preferred embodiment, the material comprises about 69% “50/17 SD” nylon, about 21% “20/1 SD” nylon, and about 10% 40 denier spandex. Further, the base material 26a is composed of materials which are generally inert and generally biocompatible. Fibers other than Nylon and spandex can be used. For example, the stretchable (spandex) component of the flexible material could comprise silicone and/or natural rubber; and the nylon could be replaced with polyethylene and/or polyurethane. The base material can be made of other types of natural fibers or man-made/industry-made fibers (or combinations thereof). For example, the other man-made/industry-made fibers can include carbon fibers, expanded polytetrafluoroethylene (commonly known by the brand name, Gore-Tex®), or other synthetic and/or conductive fibers. The top layer 26 can be made, as noted above, by weaving or knitting the fabric top layer. If the top layer is made from man-made materials, the top layer can be formed by 3D printing.

The top layer 26 further comprises upper fibers, pile, or napping 26b and lower fibers, pile, or napping 26c which extend from the upper and lower surfaces, respectively, of the base material 26a to define upper and lower napped or plush surfaces 26b,c of the top layer 26. The fibers/pile/napping 26b,c can be formed by brushing or abrading the surfaces of the base material 26a. As such, the upper and lower napped surfaces 26b,c have the same composition as the base material 26a. The upper and lower fibers/pile/napping 26b,c, depending on how they are formed, can be straight, curved, twisted, etc. Further, the fibers/pile/napping may not be uniformly shaped. The lower fibers 26c extending from the bottom of the base material 26a greatly increases the surface area of the bottom of the top layer 26 as compared to a “smooth” (i.e., un-napped) material.

The upper and lower pile or napping 26b,c can be formed by brushing the base material 26b in a single direction (preferably in a machine direction of the material). We have found that when the pile/napping is formed in this manner, when the material 22 is folded over on itself along a line generally parallel to the direction of brushing, such that upper pile contacts upper pile, the two layers of material can move relative to each other in a direction generally parallel to the fold relatively freely, and movement in this direction (generally perpendicular to the direction of brushing) is like the fabric is moving along track. On the other hand, when the fabric layers are moved relative to each other in the machine direction (i.e., parallel to the direction of brushing), when the fabric is moved in the same direction as the brushing was performed, the fabric moves freely. However, when moved against each other in a direction opposite to the direction of brushing, there is some friction, like the material is grabbing at itself.

A reinforcing layer 29 is positioned below the top layer 26, and is adhered to the top layer by an adhesive, such as an acrylate binder 29a, applied to the top side of the reinforcing layer. This reinforcing layer 29 can be made from a polyurethane film or a polyethylene film and can add some slight rigidity to the material 22, which will make the material less “floppy” and a bit easier to handle, especially when wearing gloves. Although the reinforcing agent will add some rigidity to the material 22, the reinforcing layer is stretchable, and preferably, elastic, in all directions, such that use of the reinforcing layer 29 will not diminish the stretchability or elasticity of the top layer 26. If a binder is used, the binder can be, for example, an acrylic or acrylate adhesive which will bond to the reinforcing layer and which will secure the reinforcing layer to the top layer 26. The adhesive binder 29a which adheres the reinforcing layer 29 to the top layer 26 extends into the pile 26c (or the pile 26c extends into the binder) to increase the contact surface area of the upper layer with the binder. The binder also bonds with the reinforcing layer, such that the reinforcing layer cannot easily be removed from the top layer 26.

The adherent layer 28 is secured to the bottom surface of the reinforcing layer 29. As disclosed in US20230046036, which is incorporated herein by reference, the adherent layer 28 comprises at least an adherent 28a which adheres to, but does not aggressively bond to, skin and is gentle on the skin during long term wear as well as during removal. Thus, the material 22 can be removably attached to a patient's skin, and damage to the skin during removal of the material 22 is minimized by the use of the adherent 28a. The adherent preferably comprises a silicone gel material or a hydrogel adhesive, and has viscoelastic and/or high co-adhesive properties. It can be applied to the bottom surface of the reinforcing layer 29 at a heavy (high) coat weight thickness of about 100-500 g/m², and preferably about 250-300 g/m². Such adhesives are typically composed of a very lightly cross-linked silicone elastomer whose polymer network has been swollen with silicone fluids. The silicone gel has elastic characteristics, is stretchable in any direction, and will elongate with the top layer 26 and reinforcing layer 29. That is, as the silicone gel elongates, the silicone gel layer becomes thinner. The thinning of the silicone gel layer is constant, and thus, the silicone gel layer does not get thinner in just one area, but rather thins uniformly. Thus, the silicone gel layer is colinear. The same is true for the top layer 26 and the liner 29.

Preferably, the adherent layer 28 additionally includes a binder 28b that secures the silicone gel adhesive to the bottom surface of the reinforcing layer 29. The binder 28b is preferably the same as the binder 29a. The binder bonds to both the silicone gel 28a and the reinforcing layer 29 to securely fix the adhesive layer to the reinforcing layer. If the binder 28b is used, the binder is covered by the silicone gel 28a, such that the acrylate adhesive binder 28b will not come into contact with the patient's skin during use. The reinforcing layer 29 is preferably coextensive with at least the colinear adhesive layer 28, and more preferably coextensive with the top layer 26.

The material 22 preferably is porous, in that if the upper or lower pile 26b,c of the material 22 is exposed to fluid, the fluid can travel along the material. As will be discussed below, in some of the embodiments of our ostomy appliance, the material 22 will be exposed to the accumulation volume of the pouch. In these embodiments, as discussed below, the periphery of the upper pile needs to be sealed so that output from the stoma does not travel along the barrier to a position outside of the pouch, where the output from the stoma could irritate skin. Sealing the material can be accomplished chemically or physically. If the material 22 is to be sealed mechanically, a wax, such as microcrystalline wax, available, for example, from Blended Waxes, Inc. of Oshkosh, WI, can be used. The wax can be applied to selected areas of the material 22 when the wax is in a liquid or melted state. Such a wax has a melting point substantially below that of nylon of the barrier material. Thus, the wax will not melt the upper pile 26b of the barrier. However, the liquid wax will infiltrate the spaces between the fibers of the upper pile 26b of the material 22 to adhere to the fibers. The wax will thus form a physical seal of the material 22 in the area where it is applied.

The release liner 24, as noted, is removably secured to the adherent layer 28. The release liner 24 is preferably made from a generally non-stretchable material, which is held to the adherent 28 by the natural adhesive qualities of the silicone adherent. The adhesive force of the silicone adherent is substantially less than the adhesive force of the binder, and thus, removal of the release liner 24 from the silicone adherent will not pull the silicone adherent from the top layer 26 or the reinforcing layer 29. The material for the release liner 24 is chosen by design to be a material which has low adhesion force with respect to the silicone adherent. The release liner 24 does not directly contact the top layer 26 (or reinforcing layer 29). Thus, the release liner 24 is removably adhered to the bottom of the material 22 by the silicone adherent layer 28. The release liner 24 could have a coating, for example, of fluorosilicone applied to the side of the release liner which is in contact with the silicone gel adherent. This will facilitate removal of the release liner 24 from the material 22. Additionally, the release liner could extend beyond the perimeter of the material construction 22 for ease of removal and then storing of a device when not in use. In an alternative, the device 10, 10′ could be provided with tabs or perforations, as disclosed, for example, in U.S. Pat. No. 9,427,222, which is incorporated herein by reference, to facilitate removal of the release liner 24 from the material 22.

Returning to FIG. 3A, the barrier 16, when formed from the material 22, has a thickness of about 0.042″. On the other hand, the hydrocolloid-based barrier for currently existing ostomy appliances, typically has a thickness of about 0.075″. As can be appreciated, the barrier 16 formed from the material 20 is substantially thinner than the barrier 1 of currently commercially available ostomy appliances. The barrier 16 is thus much more conformable to the shape of the patient's abdomen. Thus, the barrier 16 formed from the material 22 will substantially reduce issues of forming a seal around a stoma (even, for example, when the stoma is sunken relative to the abdominal surface or when the skin surrounding the stoma (the peristomal plane) is not flat. Further, the thinness of the material 22 makes the barrier 16 easier to cut than the material from which a typical hydrocolloid-based ostomy appliance base is made. This makes it easier to size the hole 14 in the barrier through which the stoma S extends. Further, the material presents a soft edge, and thus will not irritate the stoma.

To secure the lower envelope member 12a to the barrier 16, the flange 15b of the connection assembly 15 is bonded to the barrier 16 along a pouch/barrier junction 18. The boding of the flange 15b to the barrier 16 can be performed by ultra-sonic welding, via heat, via a chemical bonding, via adhesive, etc. If bonded chemically, a solvent is preferably used which will cause the flange 15b, which is made from nylon, to bond to the upper surface of the barrier material 22. For example, a solvent can be applied to the upper surface of the barrier material 22 in the area where the pouch/barrier junction 18 is to be formed. The pouch can then be pressed against the barrier in the area of the solvent. The solvent will then bond the pouch to the barrier to form the junction 18. In an alternative, an adhesive, such as an acrylate adhesive can be used. As discussed above in conjunction with the barrier material 22, an acrylate adhesive will infiltrate and bind to the fibers of the pile 26b. An acrylate adhesive will also bond to the material of the lower envelope member 12a to thereby securely adhere the pouch 12 to the barrier 16. Additionally, whether the lower envelope member 12a is adhered to the pouch via adhesive, solvent, or welding, the junction will create a ring which will form an annular seal surrounding the pouch and barrier openings 14, 17 which will prevent any fluids that might contact the barrier material from migrating beyond the seal ring.

As noted, the material 22 preferably is made from nylon. Nylon is not readily soluble in most solvents at room temperature, but it can be dissolved by a few specific solvents under certain conditions. For example, nylon can be solubilized by the following solvents under the noted conditions with the noted results.

| Solvent | Conditions (and Results) |
|---|---|
| Phenols, cresols, and dimethyl | Soluble in these solvents, but only at |
| formamide (DMF) | relatively high temperatures |
| | (i.e., above 55° C. |
| Formic acid | Dissolves nylon 6,6 to produce porous |
| | membranes or electrospun fibers |
| Trifluoroacetic | Dissolves nylon 6,6 |
| acid (TFA) and | to produce crystalline |
| acetone | and non-porous films |
| O-dichlorobenzene | Dissolve nylon at |
| (ODCB) or 1,2,4- | elevated temperatures |
| trichlorobenzene (TCB) | (i.e., above 100° C.) |
| Hexafluoroisopropanol (HFIP) | Dissolves nylons and polyesters at room |
| | temperature, but is expensive |

Nylon is also partially hydrolyzed when dissolved in concentrated sulfuric, nitric, or hydrochloric acid. Nylon is generally resistant to most inorganic chemicals, oils, fuels, gasoline, mineral spirits, and some alcohols. However, it can be eroded by oxidants such as hydrogen peroxide and chlorine-based decolorants. If a solvent is to be used to adhere the pouch to the barrier 16, a preferred solvent is TFA.

Bonding of the flange 15b to the barrier 16 can compress or melt the upper pile 26b of the barrier 16 in the area of the junction 18 to define an area in the barrier of reduced thickness, as seen in FIGS. 3A,B. This reduced thickness area is shown in FIGS. 3A,B as a channel in the upper pile 26c of the base 16. This bonding, whether performed by heat, ultrasonically, chemically, or adhesive, etc., makes the material 22 in the area of the junction 18 rigid and relatively stiff. Thus, to ensure that the barrier 16 remains as flexible as possible, the area of the bond between the flange 15b and the barrier is made only as large as necessary.

As seen in FIGS. 3A,B, the opening 14 is of substantially the same diameter as the opening 17 of the barrier 16, and the area 18 of the bond between the flange 15b and the barrier 16 is spaced from and is preferably concentric with the openings 14 and 17. This forms an area of the barrier 16 between the opening 17 and the bond 18 in which the barrier is still highly flexible and pliable. This allows for sizing of the openings 14, 17 as may be necessary for the stoma S without impacting the junction 18 of the flange 15b to the barrier 16. Lastly, as noted, the opening 14 has a diameter substantially equal to the diameter of the barrier opening 17. Thus, the accumulation volume 13 is defined fully by the lower and upper envelope members 12a,b. The barrier 16 itself is not exposed to the accumulation volume 13.

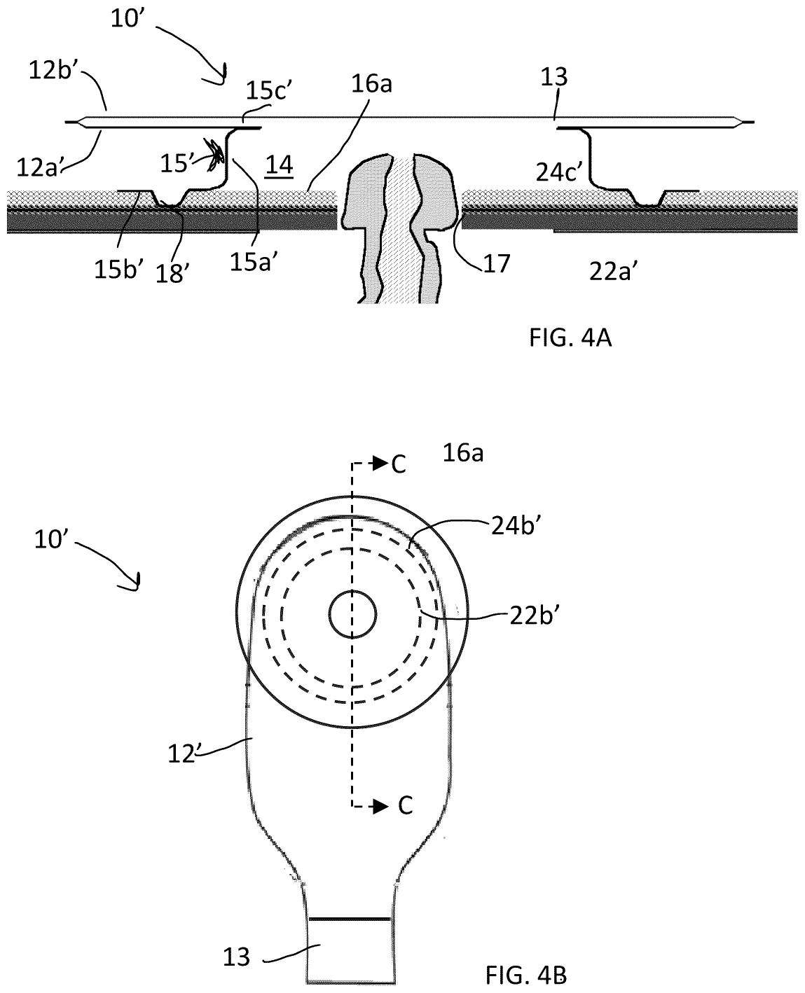

A second one-piece appliance 10′ is shown in FIGS. 4A,B. In the appliance 10′, the ostomy bag 12′ comprised of lower and upper envelope members 12a′, 12b′, respectively, which are joined together at their periphery to define an accumulation volume 13. the bag 12′ comprises a connection assembly 15′ which joins the pouch 12′ to the barrier 16. The connection assembly comprises an annular neck or wall 15a which extends from the pouch envelope lower member 12a′ and a flange 15b′ extending from a bottom of the wall 15a. However, rather than extending toward the barrier opening 17, as in the appliance 10, the flange 15b′ extends away from the junction 15c′ of the wall 15b′ to the lower envelope member 12a′ and away from the barrier opening 17. Thus, the seal 18′ formed between the flange 15b and the barrier 16 is outside of the circle defined by the wall 15b. Thus, a portion 16a of the barrier 16 is exposed to the accumulation volume 13, and could come into contact with the output from the stoma S. As described more fully below, the portion 16a of the barrier material 22 between the seal 18′ and the barrier opening 17 is sealed to substantially prevent the output of the stoma from passing through the barrier material 22 and irritating skin beneath the barrier 16.

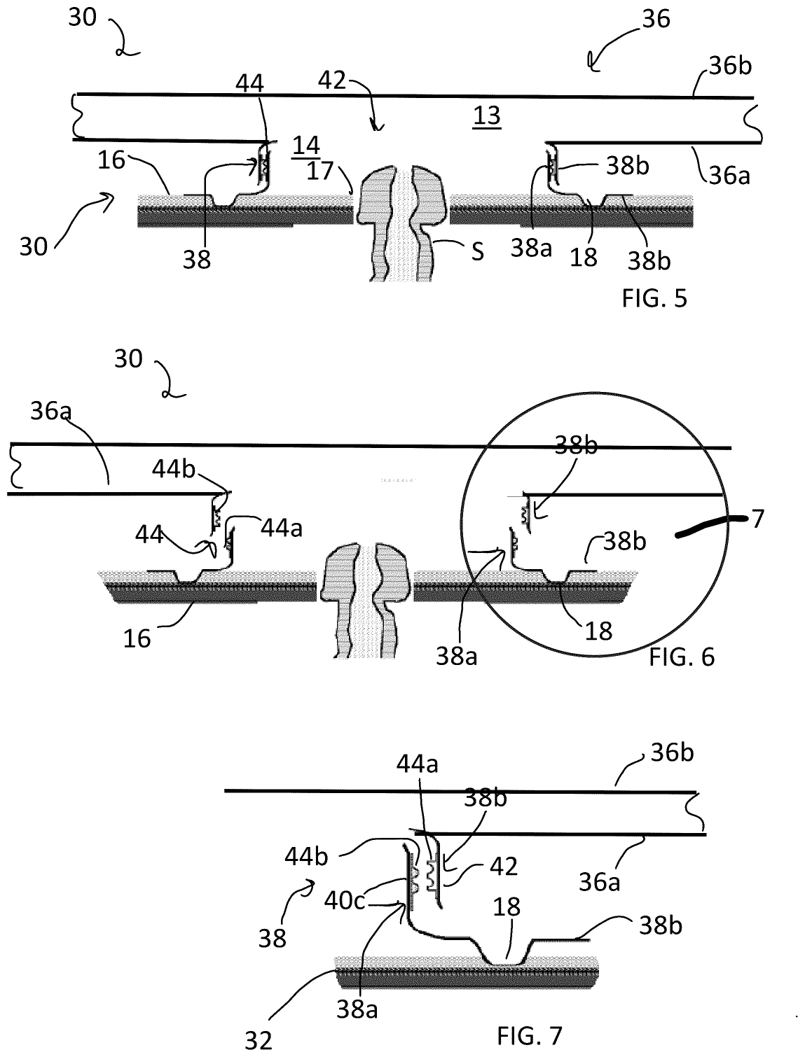

FIGS. 5-7 show a two-piece ostomy appliance 30. The ostomy appliance comprises a barrier 16 identical to the barrier of the ostomy appliances 10, 10′ and which has a barrier opening 17 through which a patient's stoma S will pass. A pouch 36 is removably securable to the barrier 16. The pouch comprises lower and upper envelope members 36a,b joined at their peripheries (not shown) to define the accumulation volume 13. A connection assembly 38 comprising a barrier connection member 38a (preferably in the form of a tube) extending from the from the barrier upper surface around the barrier opening 17 and having a flange 38b extending from an end of the connection member 38a along the barrier in a direction away from the barrier opening 17. A second connection member 38c extends from the pouch lower envelope member 36a toward the first connection member 38a. Like the first connection member, the second connection member is preferably tubular in shape. The first and second connection members 38a,c and the flange 38b can be formed from a polymer material (such as a polyolefin or ethylene-vinyl acetate), which similar to the material form which the pouch 36 is made from. The flange 28b is bonded to the barrier in the same manner as the connection assemblies 15, 15′ at the junction area 18. The connection members 38a,c are sized such that one receives the other, and both have a greater diameter that the barrier opening 17. As shown in FIGS. 5-6, the first connection member 38a is received inside of the second connection member 38c. In this arrangement, the connection members 38a,c have inner and outer mating seal rings 44a,b, respectively, with the seal rings facing each other. The first and second seal rings are formed to matingly connect with each other to form a fluid tight seal 44 between the connection members 38a,c. As seen, the two seal rings comprise corresponding and complimentarily shaped channels and ridges which allow for the two pieces to be pressed together to form the seal 40. The seal 44 can be a ZIPLOC-type seal, such as disclosed in U.S. Pat. No. 5,070,584 and RE28,969 which are incorporated herein by reference. The two piece ostomy device 36 is similar to the one piece ostomy device 10′ in that the accumulation volume 13 is defined by the base 20 with its lower envelope portion 36 and the upper envelope portion 42.

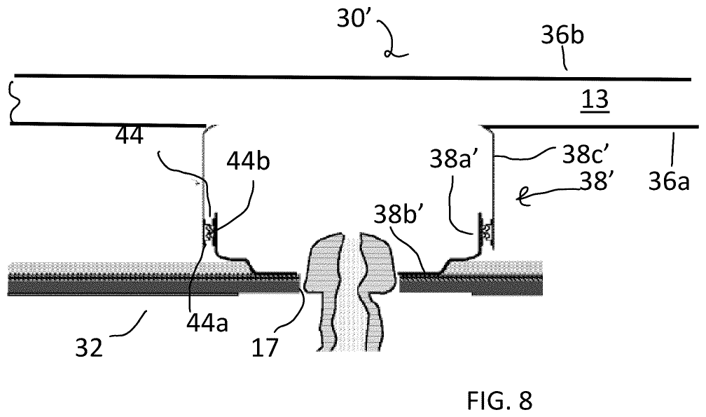

FIG. 8 shows a second two-piece ostomy appliance 30′. The two-piece ostomy appliance is substantially similar to the appliance 30 of FIGS. 5-7. To this end, the appliance 30′ comprises a pouch 36 removably connected to a barrier 16 by a connection assembly 38′. The pouch includes lower and upper envelope members 36a,b defining an accumulation volume 13; and the barrier defines a barrier opening 17. The connection assembly 38′, like the connection assembly 38, comprises a generally annular barrier connection member 38a′ which extends from the barrier 32 and a generally annual pouch connection member 38c′ which extends from the lower envelope member 36a′ of the pouch 36. The connection members 38a′,c′ are sized such that one receives the other. As shown in FIG. 8, the barrier connection member 38a′ is received inside of the pouch connection member 38c′. In this arrangement, the connection members 38a′,c′ have inner and outer mating seal rings 44a,b, respectively, with the rings facing each other. The seal rings 44a, bb are the same as the seal rings 44a,b of FIG. 7, and similarly form a seal 44 when pressed together. The barrier connection member 38a′ comprises a flange 38b′ which extends inwardly from the wall defined by the connection member 38b toward the barrier opening 34. Thus, the flange 38c′ of FIG. 8 extends in the opposite direction of the flange 38c of FIGS. 5-7. The flange 38c′ is bonded to the upper surface of the barrier 16 adjacent the barrier opening 17 over about one-half the annular width of the flange 38c′. Because the bonding of the flange to the base stiffens the base, as noted above, this forms a thinner area of base. Thus, a slight shoulder is formed in the flange 38c′ at the outer periphery of the bonded area. The outer portion of the flange 46 will then rest on the upper surface of the pile 26b until the flange merges into the wall 38a. Because the connection assembly surrounds that barrier opening 17, the accumulation volume 13 is defined fully by the lower and upper envelope members and the connection assembly 38′. Stated differently, the material 20 of the base 32 will not be exposed to the accumulation volume 13.

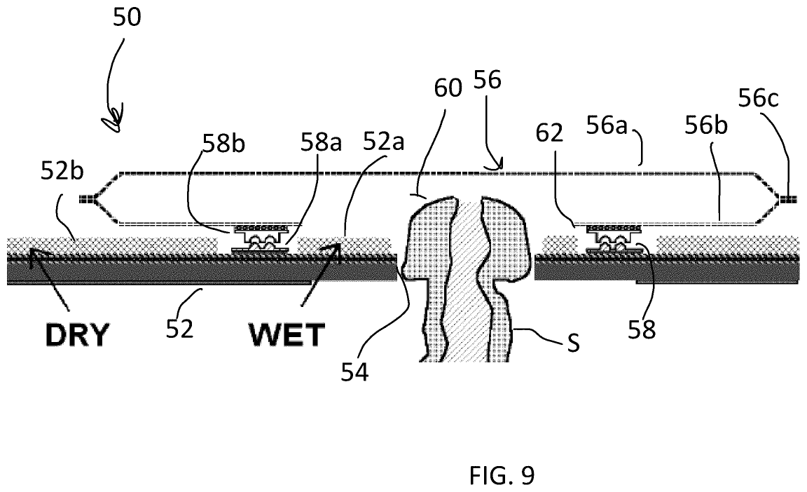

FIG. 9 shows a third two-piece ostomy appliance 50. The appliance 50 comprises a barrier 52 formed from the material 20 having an opening 54 through which the stoma S will extend in use. A pouch 56 is removably secured to the barrier 52 by means of a releasable seal 58. The barrier 52 is identical to the barrier of the prior embodiments. The pouch 56 includes an upper envelope member 56a and a lower envelope member 56b which are sealed (bonded) together at their respective peripheries, as at 56c. In combination, the upper and lower envelope members define an accumulation volume 60. The lower envelope 56b defines a pouch opening 62 through which the stoma S will extend in use.

The releasable seal 58 which releasably joins the pouch 56 to the barrier 52 comprises a first seal ring 58a bonded to the upper surface of the barrier 52 and a second seal ring 58b bonded to the lower surface of the lower envelope member 56b. The seal rings are identical in construction to the seal rings 44a,b of FIGS. 7 and 8. The first seal ring 58a has a diameter greater than the barrier opening 54 and is preferably concentric with the opening 54. Thus, the first seal ring 58a defines an inner portion 52a of the base and an outer portion 52b of the base. The second seal ring 58b preferably has an inner diameter that is the same size as, or just slightly larger than the diameter of the pouch opening 62. As with the seal 44, the first and second seal rings 58a,b comprise corresponding and complimentarily shaped channels and ridges which allow for the two pieces to be pressed together to form the seal 58.

As seen, the pouch opening 62 is substantially larger than the base/barrier opening 54. For example, the base/barrier opening 54 can have a diameter of ⅝″, whereas the pouch opening can have a diameter of 2.0″. Thus, a portion 52a of the base 52 will, in combination with the pouch 56 define the accumulation volume 60. Hence, the inner portion 52a of the base/barrier 52 can be deemed a “wet” portion of the base/barrier and the outer portion 52b can be deemed a “dry” portion of the base/barrier.

As noted, the ostomy appliances of FIGS. 5-9 are two-piece appliances. Thus, the pouch of each appliance can be removed from the base while the base is still adhered to the patient for emptying and rinsing. The pouch can then be reapplied to the base, or a new pouch can be mounted/sealed to the base. Additionally, the releasable seal of the two-piece appliance allows for a portion of the seal to be opened to release gas that may accumulate in the pouch without the need to empty the pouch. As with a typical one-piece appliance, the pouches of the one-piece appliances of FIGS. 2A,B and 4 remain securely adhered to their respective bases, and, after a period of time, will be replaced.

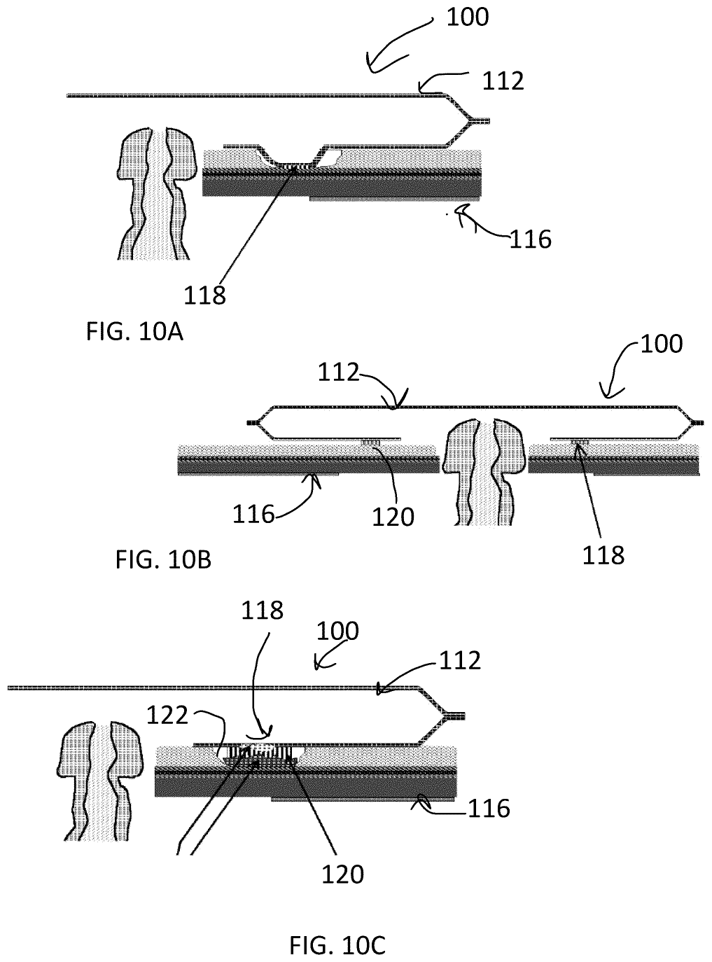

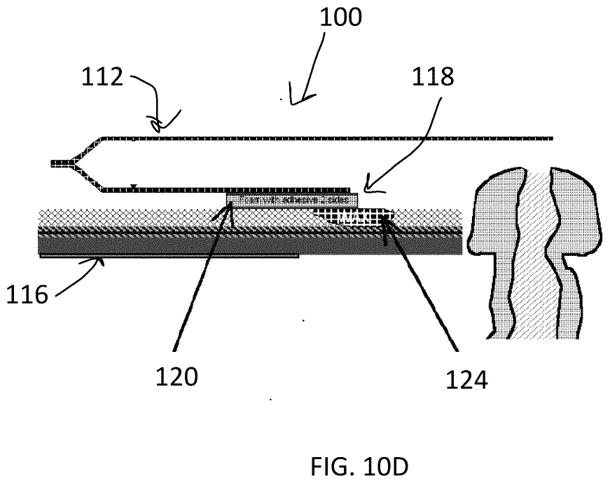

FIGS. 10A-D are sectional views of a one-piece ostomy appliance 100 in which the pouch 112 is secured directly to the barrier 116. That is, in contrast to FIGS. 3A-4B, the appliance of FIGS. 10A-D does not include a connection assembly similar to the connection wall 15, 15′. In particular, FIGS. 10A-D show four different methods of adhering or bonding the pouch 112 to the barrier 116. Briefly, the ostomy appliances 100 shown in FIGS. 10A-D are generally similar to the ostomy appliance 10 of FIG. 3A-C. To this end, the ostomy appliance 100 shown in FIGS. 10A-D includes an ostomy pouch 112 adhered to a barrier 116 at a junction 118. The ostomy pouch 112 and barrier 116 are identical to the pouch 12 and barrier 16, respectively, of FIGS. 3A-C.

In FIG. 10A, the pouch 112 is bonded to the barrier 116 using a solvent. As discussed above, in conjunction with FIGS. 3A-C, the solvent (which is preferably TFA) is applied to the upper pile 26b of the barrier material 22 in the area where the junction or bond 118 is to be formed. The solvent can, for example, be sprayed, painted, or applied in any other manner which will allow for forming a ring of solvent on the barrier material. The pouch 112 is then positioned over the barrier 116 so that their respective openings are aligned, and is then pressed against the barrier 116. In an alternative method, a ring of the solvent can also be applied to the pouch 112 which will correspond to the ring of solvent applied to the barrier 116. The solvent will melt or dissolve the upper pile 26b of the barrier material to form the channel shown in FIG. 10A. The solvent will also dissolve, melt, or otherwise soften the material of the pouch 112 to bond or weld the pouch to the barrier. Additionally, as noted above, the action of the solvent melting or dissolving the top pile 26b of the barrier material will seal barrier material 22 in the area of the junction 118 between the pouch 112 and the barrier 116.

In FIG. 10B, the pouch 112 is adhered to the barrier 116 using an annular ring of tape 120. The tape 120 is preferably a double coated polyethylene foam tape, for example, having a high performance medical grade adhesive on both faces of the tape. This tape, which can be as narrow as 1/32″, will securely adhere the pouch 112 to the barrier 116. Unlike the solvent, the use of the tape 120 will not seal pores of the barrier material.

The seal 118 in FIG. 10C is essentially a combination of the seals of FIGS. 10A, B. In the ostomy appliance of FIG. 10C, an annular channel 122 is formed the upper pile 26b of the barrier material using a solvent. The solvent will seal the barrier material in the area of the channel 122, as described above. A ring of the tape 120 is then adhered to the barrier 116 in the channel 122. The tape 122 will be secured by its adhesive. However, the solvent may also react with the tape's adhesive to further enhance the bond of the tape to the barrier materiel. The pouch 112 is then secured to the opposite (top) side of the tape 120. The tape's adhesive will secure the pouch to the tape. However, a second application of solvent can be applied to the pouch 112 and/or the tape 120. This second application of the solvent will further enhance the bond of the tape to the pouch.

The ostomy appliance of FIG. 10D relies on a ring of double sided tape 120 to adhere the pouch 112 to the barrier 116. However, as seen, the tape 120 in FIG. 10D is wider than the tape of FIGS. 10B,C. This wider tape provides for a larger contact area with both the barrier and the pouch, and thus will better secure the pouch to the barrier. In addition, the junction 118 of FIG. 10D is provided with a ring of wax 124 which is applied to the upper pile 26b of the barrier material. The wax can be a microcrystalline wax, such as is available from Blended Waxes, Inc. of Oshkosh, WI. The wax 124 is applied to the barrier material when melted. The wax has a melting point substantially below that of nylon (the material from which the barrier is made). Thus, the wax will not melt the barrier's upper pile 26b. However, the liquid wax will infiltrate the spaces between the fibers of the upper pile 26b of the material 22 to adhere to the fibers. The wax will thus form a physical seal of the material 22 in the area where it is applied. As seen in FIG. 10D, the wax 124 will form an annular ring having a depth generally equal to the depth of the upper pile 26b and an upper surface which is level with the upper surface of the barrier material. The wax ring and the tape define respective inner and outer diameters. The inner diameter of the tape is between the inner and outer diameters of the wax ring and the outer diameter of the tape is greater than the outer diameter of the wax ring. Thus, the tape will extend over a portion of the wax ring as will as over the barrier immediately outside of the wax ring, as seen in FIG. 10D. In this version, the was will form the seal of the barrier material that is formed by the solvent in the ostomy appliances of FIGS. 10A and 10C. In an alternative, a silicone can be used in lieu of the wax.

Although the various methods of attaching the pouch to the barrier are described in connection with a one-piece ostomy appliance, they are applicable to the two-piece appliances as well. For example, the various methods described above can be used to secure the lower portions 38a of the connection assemblies of the appliances of FIGS. 5A-8B and for connecting the seal ring 58a to the barrier material in the appliance 50 of FIG. 9.

As various changes could be made in the above constructions without departing from the scope of the invention, it is intended that all matter contained in the above description or shown in the accompanying drawings shall be interpreted as illustrative and not in a limiting sense. These examples are merely illustrative.

1. An ostomy appliance comprising a barrier and a pouch which is adhered to the barrier;
said pouch comprising an upper envelope member and a lower envelope member, which in combination define at least a portion of an accumulation volume;
the lower envelope member defining a pouch opening;
said barrier defining a barrier opening aligned with said pouch opening; said pouch and barrier openings being adapted to fit about a stoma in use;
the barrier being made of a barrier material comprising:
a top layer; the top layer comprising a base material, an upper pile extending from an upper surface of said base material, and a lower pile extending from a lower surface of said base material; said base material being flexible and stretchable;
a reinforcing layer, said reinforcing layer being secured to said top layer by a first binder which will bond to said reinforcing layer; said binder preferably being an acrylate binder; said lower pile of said top layer extending into said binder; said reinforcing layer preferably being formed from a polyurethane film or a polyethylene film; and
an adherent layer comprising a silicone gel, said silicone gel being operatively bound to said reinforcing layer by a second binder; said binder being selected to be a binder, such as an acrylate binder, which will bond to the silicone gel and to the reinforcing layer.

2. The ostomy appliance of claim 1 wherein said ostomy appliance is a one-piece ostomy appliance; said upper and lower envelope members being joined together at respective peripheries; said appliance comprising a connection assembly; said connection assembly comprising an tubular member extending from said lower envelope member about said pouch opening and a flange extending from said tubular member; said flange extending across a portion of said barrier; said flange being secured to said barrier along a junction area; said junction area defining a ring concentric with said openings of said lower envelope and said barrier and having a diameter greater than the diameter of said barrier opening.

3. The ostomy appliance of claim 2 wherein said flange extends toward said barrier opening, whereby the ring defined by said junction area defines a diameter smaller than the diameter of said tubular member, said flange being sized such that an inner edge of said flange defines a perimeter of substantially the same size as said barrier opening.

4. The ostomy appliance of claim 2 wherein said flange extends away from said barrier opening, whereby the ring defined by said junction area defines a diameter greater than the diameter of said tubular member, and the upper surface of said barrier inside of said connection member is exposed to said accumulation volume.

5. The ostomy appliance of claim 1 wherein said pouch is adhered to said barrier by means of a solvent, adhesive tape, or a combination thereof.

6. The ostomy appliance of claim 5 wherein said solvent seals pores of said barrier material in the area of said junction.

7. The ostomy appliance of claim 1 including a wax or silicone ring formed in an annular channel in said barrier about said barrier opening, wherein said pouch is secured to said barrier by an adhesive tape; said adhesive tape defining a ring having an inner diameter smaller than an outer diameter of said wax or silicone ring and an outer diameter greater than the outer diameter of said wax or silicone ring, whereby, said tape covers a portion of said was ring and extends over a portion of said barrier adjacent said wax or silicone ring.

8. The ostomy appliance of claim 1 wherein said ostomy appliance is a two-piece appliance and wherein said pouch is detachable and reappliable to the barrier.

9. The ostomy appliance of claim 8 comprising a connection assembly which is operable to is removably connect said pouch to said barrier; said connection assembly comprising a first seal ring operably connected to said barrier and a second seal ring operably connected to said lower envelope member of said pouch.

10. The ostomy appliance of claim 9 wherein connection assembly comprises:
a first annular wall and a second annular wall;
said first annular wall being adhered to, and extending from, barrier, said first annular wall surrounding said barrier opening; said first annular wall having flange at a first end thereof which is adhered to said barrier;
said second annular wall being adhered to, and extending from said envelope lower member, said second annular wall defining said pouch opening (14);
said first and second annular walls being sized such that one is received within the other;
said first seal ring being received on an inner surface of one of said first and second annular walls and said second sealing ring being received on an outer surface of the other of said first and second annular walls, whereby said first and second sealing rings are aligned with, and face, each other such that they can be pressed together to form a seal and connect said pouch to said barrier.

11. The ostomy appliance of claim 10 wherein said flange of said first annular wall extends away from said barrier opening, whereby said upper surface of said barrier is exposed to said accumulation volume.

12. The ostomy appliance of claim 10 wherein said flange of said first annular wall extends toward said barrier opening, an inner edge of said flange being proximate said barrier opening, whereby said upper surface of said barrier is substantially not exposed to said accumulation volume.

13. The ostomy appliance of claim 9 wherein said first seal ring is adhered to said barrier about said barrier opening and said second seal ring is adhered to said lower envelope member of said pouch about said pouch opening; said first and second seal rings facing each other such that when connected said pouch opening is aligned with said barrier opening.

14. The ostomy appliance of claim 9 wherein said first and second seal rings defines a zipper-type seal.

15. The ostomy appliance of claim 9 wherein the barrier is reusable and the pouch is disposable.

16. The ostomy appliance of claim 9 wherein said flange of said first annual wall pouch is adhered to said barrier by means of a solvent, adhesive tape, or a combination thereof.

17. The ostomy appliance of claim 16 wherein said solvent seals pores of said barrier material in the area of said junction between said flange and said barrier.

18. The ostomy appliance of claim 9 including a wax or silicone ring formed in an annular channel in said barrier about said barrier opening, wherein said flange of said first annular wall is secured to said barrier by an adhesive tape; said adhesive tape defining a ring having an inner diameter smaller than an outer diameter of said wax or silicone ring and an outer diameter greater than the outer diameter of said wax or silicone ring, whereby, said tape covers a portion of said was ring and extends over a portion of said barrier adjacent said wax or silicone ring.
